(19) United States
(12) Patent Application Publication
(10) Pub. No.: US 2025/0076329 A1
(43) Pub. Date: Mar. 6, 2025

(54) PREPROCESSING APPARATUS AND ANALYSIS SYSTEM

(71) Applicant: SHIMADZU CORPORATION, Kyoto-shi (JP)

(72) Inventors: Atsushi SAKAMOTO, Kyoto-shi (JP); Hironobu YAMADA, Kyoto-shi (JP); Motomu HINOOKA, Kyoto-shi (JP)

(21) Appl. No.: 18/823,251

(22) Filed: Sep. 3, 2024

(30) Foreign Application Priority Data
Sep. 5, 2023 (JP) 2023-143775

(51) Int. Cl. G01N 35/02; G01N 35/00; G01N 35/10

(52) U.S. Cl. CPC G01N 35/02; G01N 35/00722; G01N 35/1065

(57) ABSTRACT

A preprocessing apparatus includes a preprocessing module, first and second mounting areas, a central control device, and first and second communication units. The preprocessing module transmits first attribute information to the central control device via the first communication unit when it is mounted in the first mounting area. The preprocessing module transmits the first attribute information to the central control device via the second communication unit when the preprocessing module is mounted in the second mounting area. The central control device identifies that the preprocessing module is mounted in the first mounting area when the first attribute information is received from the first communication unit and identifies that the preprocessing module is mounted in the second mounting area when the first attribute information is received from the second communication unit.

### CROSS-REFERENCE TO RELATED APPLICATIONS

This application claims priority under 35 U.S.C. § 119 to Japanese Patent Application No. 2023-143775 filed on Sep. 5, 2023, the entire disclosure of which is incorporated herein by reference in its entirety.

### BACKGROUND OF THE INVENTION

### Field of the Invention

The present disclosure relates to a preprocessing apparatus and an analysis system.

### Description of the Related Art

The following description sets forth the inventor's knowledge of the related art and problems therein and should not be construed as an admission of knowledge in the prior art.

Conventionally, a preprocessing apparatus and an analysis system including a preprocessing apparatus are known. The preprocessing apparatus performs preprocessing on cells contained in a sample, such as, e.g., a culture medium. An analysis system is equipped with a preprocessing apparatus and an arm for moving a sample container containing a sample. The arm is configured to transfer the sample container to a location appropriate for the application. After completion of the preprocessing, the pretreated sample is supplied to an analysis device, such as, e.g., a liquid chromatograph mass spectrometer, included in an analysis system.

Japanese Unexamined Patent Application Publication No. 2019-174369 discloses a preprocessing apparatus equipped with an arm. The arm transports the sample container gripped by a gripper to the preprocessing apparatus.

On the deck of the preprocessing apparatus, a plurality of preprocessing mechanisms for performing preprocessing is mounted. The preprocessing includes various processes such as centrifugation, liquid removal, reagent supply, agitation, and extraction. Each preprocessing mechanism mounted on the deck performs processing corresponding to one of several types of preprocessing. The arm transfers the sample container to the preprocessing mechanism where desired preprocessing is performed, in accordance with the scheduled preprocessing sequence.

By the way, the space available on the deck of the preprocessing apparatus is limited, while the types of preprocessing are extensive. For this reason, a preprocessing apparatus is not equipped with a preprocessing mechanism for each of all types of preprocessing. Conventionally, in cases where a preprocessing apparatus owned by a user was not equipped with a preprocessing mechanism corresponding to the desired preprocessing, the user had to ask a specialized contractor to modify the preprocessing apparatus.

In this case, the specialized contractor needs to disassemble the hardware of the preprocessing apparatus, install the desired preprocessing mechanism in the preprocessing apparatus, and modify the software of the preprocessing apparatus so that the preprocessing apparatus can recognize the new preprocessing mechanism. For this reason, there has conventionally been a problem with the user bearing the cost burden of modifying the preprocessing apparatus.

### SUMMARY OF THE INVENTION

Preferred embodiments of the present disclosure have been developed in view of the above-mentioned and/or other problems in the related art. The preferred embodiments of the present disclosure can significantly improve upon existing methods and/or apparatuses.

The present disclosure has been made to solve the above problems, and the purpose of the present disclosure is to enable the easy implementation of additions and/or modifications of preprocessing functions.

A preprocessing apparatus according to the present disclosure is a preprocessing apparatus for preprocessing a sample contained in a sample container.

The preprocessing apparatus includes:
a first preprocessing module having a first function related to the preprocessing, the first preprocessing module being configured to output first attribute information for identifying the first function;
an arm configured to transfer the sample container to the first preprocessing module;
a mounting unit having a first mounting area and a second mounting area, the first mounting area and the second mounting area each being configured to mount the first preprocessing module therein;
a central control device;
a first communication unit arranged in the first mounting area; and
a second communication unit arranged in the second mounting area,
wherein the first preprocessing module transmits the first attribute information to the central control device via the first communication unit when the first preprocessing module is mounted in the first mounting area,
wherein the first preprocessing module transmits the first attribute information to the central control device via the second communication unit when the first preprocessing module is mounted in the second mounting area, and
wherein the central control device is configured to identify that the first preprocessing module is mounted in the first mounting area when the first attribute information is received from the first communication unit, and
identify that the first preprocessing module is mounted in the second mounting area when the first attribute information is received from the second communication unit.

The above and other objects, features, aspects and advantages of the present disclosure will become apparent from the following detailed description of the present disclosure understood in connection with the accompanying drawings.

The above and/or other aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspects or features of other embodiments where applicable. The descriptions of aspects, features and/or advantages of particular embodiments should not be construed as limiting other embodiments or the claims.

### BRIEF DESCRIPTION OF THE DRAWINGS

The preferred embodiments of the present disclosure are shown by way of example, and not limitation, in the accompanying figures.

FIG. 1 is a front view showing a schematic configuration of an analysis system.

FIG. 2 is a see-through perspective view showing a schematic configuration of a preprocessing apparatus.

FIG. 3 is a perspective view showing the configuration of a preprocessing module.

FIG. 4 is a side view of a mounting area and a preprocessing module.

FIG. 5 is a diagram showing the corresponding relation between the mounting areas provided on the deck and the connectors provided on the central board.

FIG. 6 is a block diagram showing the configuration of the preprocessing apparatus.

FIG. 7 is a flowchart including processing performed by a central control device and a preprocessing module.

FIG. 8 is a diagram showing table data stored in a memory of a central control device.

FIG. 9 is a conceptual diagram showing the configuration of Modification 1 regarding the signal lines connecting the mounting areas to the central board.

FIG. 10 is a block diagram showing the configuration of Modification 1 regarding the signal lines connecting between the mounting areas and the central board.

FIG. 11 is a diagram showing the corresponding relation between the number of SCI signal lines and the number of Enable signal lines according to Modification 1.

FIG. 12 is a block diagram showing the configuration according to Modification 2.

FIG. 13 is a diagram showing the relation between the preprocessing module and the mounting area according to Modification 3.

FIG. 14 is a diagram showing the mounting reference position and the arm reference position set in the preprocessing module.

FIG. 15 is a block diagram showing the configuration of the module board according to Modification 3.

FIG. 16 is a conceptual diagram for explaining the information that is to be transmitted from the preprocessing module to the central control device.

### DETAILED DESCRIPTION OF THE PREFERRED EMBODIMENTS

In the following paragraphs, some preferred embodiments of the present disclosure will be described by way of example and not limitation. It should be understood, based on this disclosure, that various other modifications can be made by those skilled in the art based on these illustrated embodiments.

Hereinafter, some embodiments of the present disclosure will be described with reference to the attached figures. Note that the same or equivalent part in the figures is assigned by the same reference symbol, and the description will not be repeated.

### <Schematic Configuration of Analysis System 100>

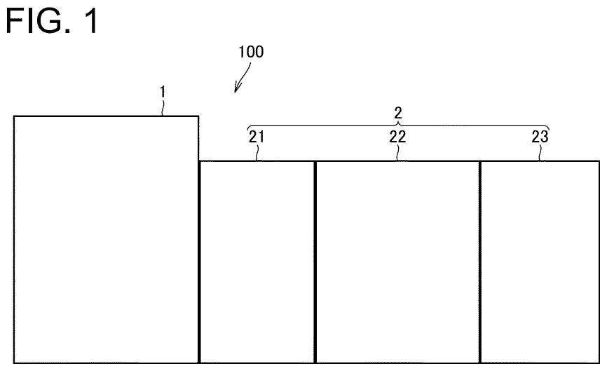

FIG. 1 is a front view showing a schematic configuration of an analysis system 100. The analysis system 100 automatically performs preprocessing and analysis on an analysis target. In this embodiment, the analysis target is a cell contained in a liquid sample, such as a culture medium, and more specifically, a fungus.

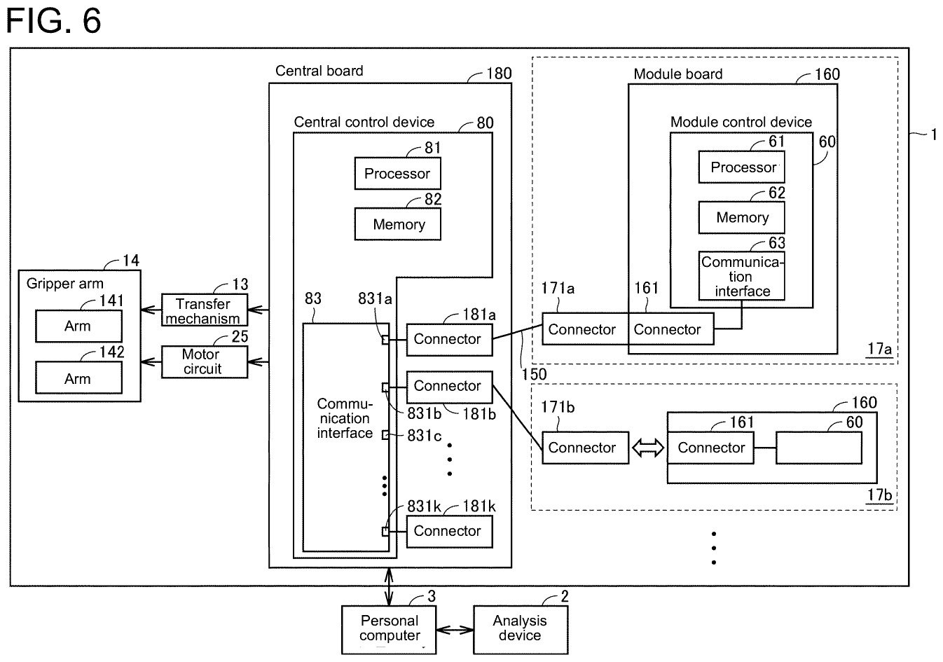

The analysis system 100 includes a preprocessing apparatus 1 and an analysis device 2. The analysis system 100 further includes a personal computer 3 (see FIG. 6). The personal computer 3 is communicatively connected to the preprocessing apparatus 1 and the analysis device 2.

The preprocessing apparatus 1 is equipped with a plurality of preprocessing mechanisms. The preprocessing apparatus 1 operates those mechanisms to perform preprocessing on a liquid sample.

The analysis device 2 includes an autosampler 21, a liquid chromatograph apparatus 22, and a mass spectrometer 23. The autosampler 21, the liquid chromatograph apparatus 22, and the mass spectrometer 23 are connected by piping and wiring, not illustrated in the figure. The autosampler 21 is a device for sampling a liquid sample (hereinafter also referred to simply as “sample”) from a sample container (labware). The preprocessed liquid sample is introduced into the liquid chromatograph apparatus 22 via the autosampler 21. Thereafter, the sample is analyzed by the liquid chromatograph apparatus 22 and the mass spectrometer 23.

In FIG. 1, the autosampler 21 is included in the concept of the analysis device 2, but the autosampler 21 may be excluded from the concept of the analysis device 2. The liquid chromatograph apparatus 22 and the mass spectrometer 23 are each just one example of an analysis device for analyzing an analysis target. Other analysis systems may be adopted for the analysis system 100.

### <Schematic Configuration of Preprocessing Apparatus 1>

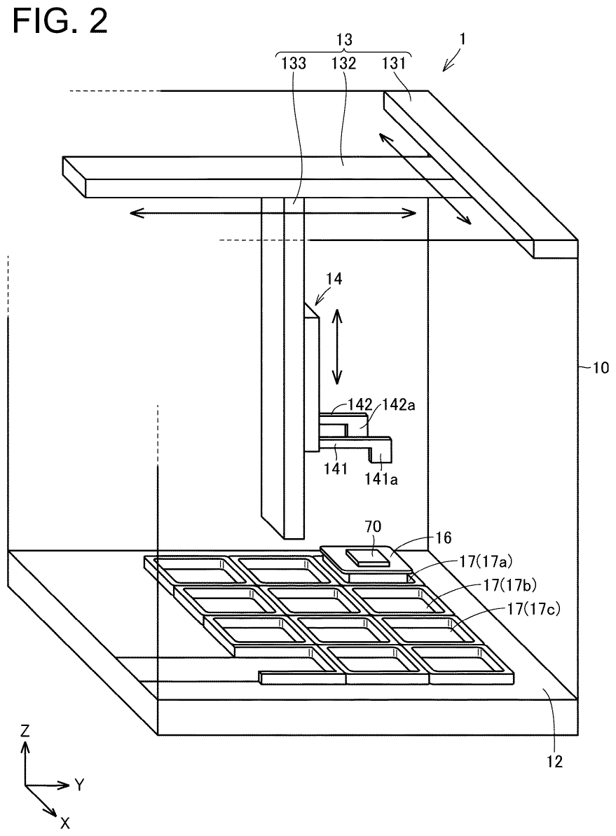

FIG. 2 is a see-through perspective view showing the schematic configuration of the preprocessing apparatus 1. As shown in FIG. 2, the preprocessing apparatus 1 has a deck 12 having a top surface parallel to the X-Y plane. The entire space above the deck 12 is enclosed by a housing 10. The deck 12 is provided with a plurality of mounting areas (deck holes) 17. For example, FIG. 2 illustrates an example in which eleven mounting areas 17 are provided in a matrix. In the mounting area 17, a preprocessing module 16, which is one example of the preprocessing mechanism, is mounted in a removable manner.

In FIG. 2, one of the preprocessing modules 16 to be mounted in each of the plurality of mounting areas 17 is illustrated as a representative example. The number of mounting areas 17 provided on the deck 12 is not limited to 11 (eleven). The number of the mounting areas 17 provided on the deck 12 may be 1 (one), or any number of two or more. The deck 12 is one example of the mounting unit having a first mounting area and a second mounting area.

The content of the preprocessing performed by the preprocessing module 16 differs depending on the type of the preprocessing module 16. Before using the preprocessing apparatus 1, the user selects one or more of the preprocessing modules 16 according to the desired preprocessing from among the various types of preprocessing modules 16. The user mounts each of the selected preprocessing modules 16 in any of the multiple mounting areas 17. A sample container 70 is arranged in one of the plurality of preprocessing modules 16. The sample container 70 contains a sample to be subjected to preprocessing. The preprocessing module 16 performs preprocessing on the sample in the sample container 70.

Above the deck 12, a transfer mechanism 13 is provided. The transfer mechanism 13 includes an X-axis guide rail 131 extending in the X-axis direction, a Y-axis guide rail 132 extending in the Y-axis direction, and a Z-axis guide rail 133 extending in the Z-axis direction.

The X-axis guide rail 131 is attached to the upper portion of the housing 10. The Y-axis guide rail 132 is attached to the X-axis guide rail 131 so that it can move along the X-axis guide rail 131 in the X-axis direction. The Z-axis guide rail 133 is attached to the Y-axis guide rail 132 so that it can move along the Y-axis guide rail 132 in the Y-axis direction. The gripper arm 14 is attached to the Z-axis guide rail 133 to be movable in the Z-axis direction.

The transfer mechanism 13 further includes a plurality of drive mechanisms (not illustrated). The plurality of drive mechanisms is configured to move the Y-axis guide rail 132 in the X-axis direction, the Z-axis guide rail 133 in the Y-axis direction, and the gripper arm 14 in the Z-axis direction. This causes the gripper arm 14 to move in three axes directions, i.e., the X-axis direction, the Y-axis direction, and the Z-axis direction, within the housing 10.

The gripper arm 14 transfers the sample container 70 from one preprocessing module 16 to another preprocessing module 16 according to a predetermined preprocessing sequence. The gripper arm 14 has arms 141 and 142. The arm 141 includes a gripper 141a, and the arm 142 includes a gripper 142a. The gripper arm 14 grips the sample container 70 containing a sample with a pair of grippers 141a and 142a and transfers the sample container 70 to the preprocessing module 16.

### <Configuration of Preprocessing Module 16 and Mounting Area 17>

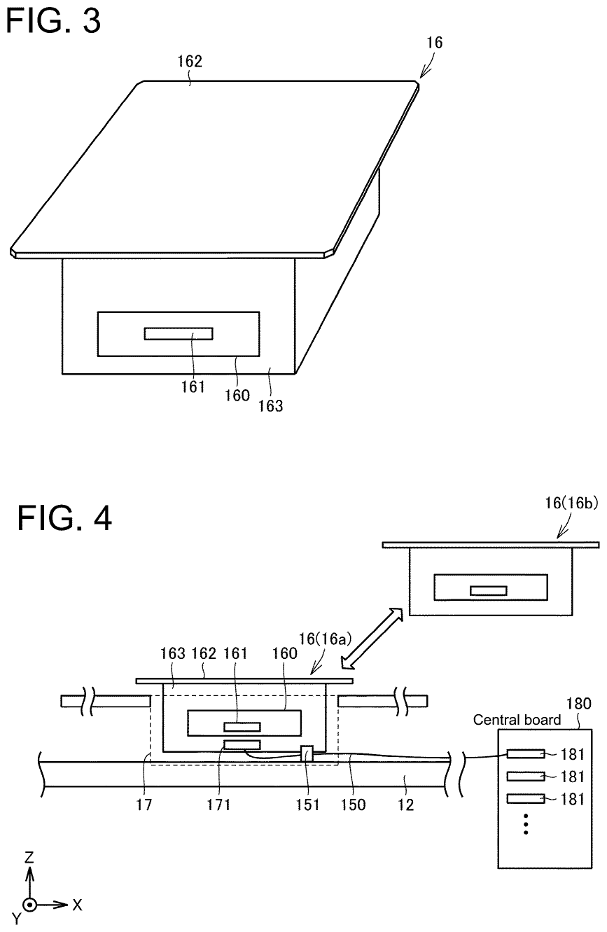

FIG. 3 is a perspective view showing the configuration of the preprocessing module 16. FIG. 4 is a side view of the mounting area 17 and the preprocessing module 16. As shown in FIG. 3, the preprocessing module 16 includes a top plate 162 on which the sample container 70 is to be placed and a support base 163 that supports the top plate 162. The support base 163 is provided with a module board 160. A connector 161 is attached to the module board 160.

Note that the connector 161 may be arranged on either the side or the bottom of the preprocessing module 16. Further, the module board 160 may be mounted inside the preprocessing module 16. However, the connector 161 must be arranged so that it can be easily connected to wiring 150.

The preprocessing module 16 includes hardware that performs preprocessing functions and a control system corresponding to the hardware. For example, the preprocessing module 16 having a stirring function is provided with hardware necessary for stirring and a module board 160 for controlling the stirring.

The preprocessing module 16 has a shape corresponding to the preprocessing function. FIG. 3 shows one example of the preprocessing module 16 in various shapes in a simplified form. Further, FIG. 4 shows preprocessing modules 16a and 16b as examples of preprocessing modules 16 having various shapes and preprocessing functions. Each of the preprocessing modules 16a and 16b is provided with a module board 160 and a connector 161. Hereafter, the term “preprocessing module 16” should be understood as a generic term for preprocessing modules having various shapes and preprocessing functions, including the preprocessing modules 16a and 16b.

The preprocessing module 16 is mounted in the mounting area 17. A connector 171 is fixed to a predetermined position in the mounting area 17. The user pushes the preprocessing module 16 downward from above the mounting area 17. With this, the connector 161 on the preprocessing module 16 is connected to the connector 171 fixed to the mounting area 17. As a result, the preprocessing module 16 is fixed to a predetermined position in the mounting area 17. At this time, the top plate 162 is positioned above the mounting area 17. The user can mount the preprocessing module 16 in the mounting area 17 by following this procedure.

Note that when placing the preprocessing module 16 in the mounting area 17, the connector 161 of the preprocessing module 16 may be connected to the connector 171 fixed in the mounting area 17 in any manner.

The user can remove the preprocessing module 16a, which is mounted in the mounting area 17, from the connector 171 and mount the preprocessing module 16b in the mounting area 17. In this case, the user repeats the above procedure using the preprocessing module 16b. With this, the user can change the function of the preprocessing performed in the mounting area 17. The preprocessing module 16a is one example of the first preprocessing module, and the preprocessing module 16b is one example of the second preprocessing module.

The wiring 150 leading to the central board 180 is connected to the connector 171 in the mounting area 17. The wiring 150 is secured to the deck 12 with a stopper 151.

The central board 180 is provided with a plurality of connectors 181. The wiring 150 is connected to one of the plurality of connectors 181. The wiring 150 includes communication lines for communication and power lines that supply power from the central board 180 to the preprocessing module 16. The module board 160 communicates with the central board 180 through the connectors 161 and 171 and the wiring 150.

The central board 180 transmits various commands, including those related to preprocessing, to the module board 160. The module board 160 executes various types of processing corresponding to the commands. The central board 180 requests attribute information from the module board 160. The attribute information enables the central board 180 to identify the attribute (preprocessing function) of the preprocessing module 16. The module board 160 transmits attribute information to the central board 180 upon request. The central board 180 identifies the attributes of the preprocessing module 16 based on the attribute information.

In the mounting area 17, various types of preprocessing modules 16 with different preprocessing functions, such as a drying preprocessing module, an agitation preprocessing module, a cooling preprocessing module, and a barcode reader preprocessing module, are mounted. Other types of preprocessing modules include: a preprocessing module that uses a camera to check whether a given chip is attached to the sample container 70; a preprocessing module that collects magnetic beads from the sample in the sample container 70; a preprocessing module that rotates a rack on which many sample containers 70 are placed; and a standby preprocessing module that keeps the sample containers 70 on standby. The preprocessing modules 16a and 16b shown in FIG. 4 may be any of the preprocessing modules exemplified above.

Note that the standby preprocessing module functions only to keep the sample container 70 in the position of the standby preprocessing module. Therefore, the module board 160 in the standby preprocessing module does not perform any operations related to preprocessing. However, the module board 160 in the standby preprocessing module communicates with the central board 180 and transmits the attribute information to the central board 180. Based on the attribute information, the central board 180 identifies the communication partner as a standby preprocessing module.

The user selects a preprocessing module 16 that suits their purpose from among several types of preprocessing modules 16 and mounts the selected preprocessing module 16 in the mounting area 17. As described above, the user can remove the preprocessing module 16 mounted in the mounting area 17 from the connector 171 and mount another type of preprocessing module 16 in the mounting area 17.

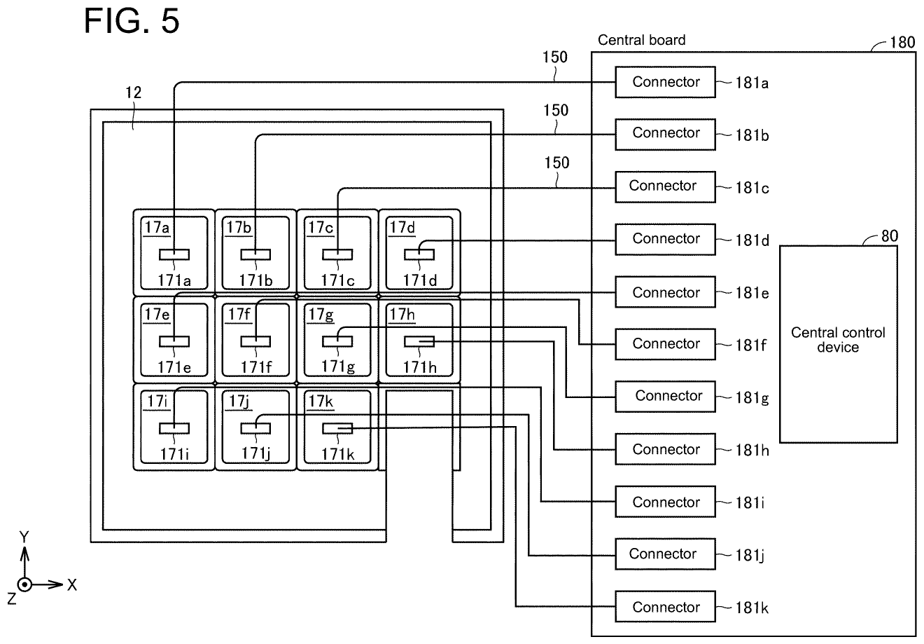

FIG. 5 is a diagram showing the corresponding relation between the mounting areas 17 provided on the deck 12 and the connectors provided on the central board 180. In FIG. 5, the symbols 17a to 17k are used to distinguish each of the plurality of mounting areas 17 on the deck 12. Similarly, the symbols 171a to 171k are used to distinguish the connectors 171 provided in each of the plurality of mounting areas 17. Similarly, the symbols 181a to 181k are used to distinguish each of the plurality of connectors 181 provided on the central board 180.

Hereinafter, the term “mounting area 17” should be understood as a generic term for the mounting areas 17a to 17k, the term “connector 171” should be understood as a generic term for the connectors 171a to 171k, and the term “connector 181” should be understood as a generic term for the connectors 181a to 181k.

The mounting areas 17a to 17k are the same in size. Further, the connectors 171 arranged in each of the mounting areas 17a to 17k are also the same in shape. The connectors 171 are fixed at the same position in each of the mounting areas 17a to 17k.

The central board 180 is provided in the preprocessing apparatus 1. The central board 180 includes a central control device 80 that controls the preprocessing apparatus 1 and the connectors 181a to 181k. The connector 181a is connected to the connector 171a arranged in the mounting area 17a by wiring 150. The connector 181b is connected to the connector 171b arranged in the mounting area 17b by wiring 150. Similarly, the connectors 181c to 181k are connected to the connectors 171c to 171k by wiring 150.

Therefore, the connectors 181a to 181k on the central board 180 correspond to the mounting areas 17a to 17k, respectively. The connection relation between the connectors 171 in each of the mounting areas 17a to 17k and the connectors 181 on the central board 180 is fixed. Therefore, the preprocessing module 16 mounted in the mounting area 17a communicates with the central control device 80 via the connector 181a on the central board 180, and the preprocessing module 16 mounted in the mounting area 17b communicates with the central control device 80 via the connector 181b on the central board 180. In this way, in this embodiment, the mounting area 17 of the preprocessing module 16 that communicates with the central control device 80 via the connector 181 is identified by the location of the connector 181.

The mounting area 17a is one example of a first mounting area, and the mounting area 17b is one example of a second mounting area. The connector 171a is one example of a first communication unit, and the connector 171b is one example of a second communication unit. The connector 181a is one example of a first central connector, and the connector 181b is one example of a second central connector.

### <Block Diagram of Preprocessing Apparatus 1>

FIG. 6 is a block diagram showing the configuration of the preprocessing apparatus 1. As shown in FIG. 6, the preprocessing apparatus 1 includes a transfer mechanism 13, a gripper arm 14, a motor circuit 25, and a central board 180. The central control device 80, the transfer mechanism 13, the gripper arm 14, and the motor circuit 25 constitute the arm mechanism. In each of the plurality of mounting areas 17 (see FIG. 2) of the preprocessing apparatus 1, a preprocessing module 16 of the type selected by the user is mounted. In FIG. 6, the mounting areas 17a and 17b are illustrated.

The transfer mechanism 13 includes the X-axis guide rail 131, the Y-axis guide rail 132, and the Z-axis guide rail 133. The transfer mechanism 13 further includes a plurality of drive mechanisms (not illustrated). The central control device 80 controls the plurality of drive mechanisms to move the Y-axis guide rail 132 in the X-axis direction, the Z-axis guide rail 133 in the Y-axis direction, and the gripper arm 14 in the Z-axis direction in the Z-axis guide rail 133. As a result, the gripper arm 14 moves in the housing 10 in the X-, Y-, and Z-axis directions.

The motor circuit 25 is one example of a drive mechanism that drives the arms 141 and 142 of the gripper arm 14. As a motor, for example, a stepping motor is used. The central control device 80 adjusts the spacing between the arms 141 and 142 by controlling the motor circuit 25.

The central board 180 includes the central control device 80 and connectors 181a to 181k. The central control device 80 includes a processor 81, a memory 82, and a communication interface 83. The central control device 80 is communicatively connected to the analysis device 2 via, for example, a personal computer 3.

The processor 81 is typically a CPU (Central Processing Unit), an MPU (Multi-Processing Unit), or the like. The processor 81 reads out and executes the program stored in the memory 82 to realize various processing of the preprocessing apparatus 1. The memory 82 is configured to include, for example, a RAM (Random Access Memory), a ROM (Read Only Memory), a flash memory, and a hard disk. The memory 82 stores various data such as preprocessing settings, position information (x-coordinate, y-coordinate, and z-coordinate) of the mounting areas 17a to 17k, in addition to programs executed by the processor 81.

The communication interface 83 includes communication ports 831a to 831k. The communication ports 831a to 831k are connected to the connectors 181a to 181k, respectively. As already described, the connectors 181a to 181k are connected to the connectors 171 in the mounting areas 17a to 17k, respectively. The module board 160 of the preprocessing module 16 is connected to each connector 171 of the mounting areas 17a to 17k.

Therefore, the central control device 80 identifies the mounting area 17 of the preprocessing module 16, which is the communication partner, based on the communication ports 831a to 831k used for communication. For example, the central control device 80 identifies that the communication destination via the communication port 831a is the preprocessing module 16 in the mounting area 17a. Similarly, the central control device 80 identifies that the communication destination via the communication port 831b is the preprocessing module 16 in the mounting area 17b.

As shown in FIG. 6, the module board 160 includes a module control device 60 and a connector 161. The module control device 60 includes a processor 61, a memory 62, and a communication interface 63. The connector 161 is connected to the communication interface 63. The processor 61 is typically a CPU (Central Processing Unit), an MPU (Multi-Processing Unit), or the like. The connector 161 is one example of a module connector.

The processor 61 reads out and executes a program stored in the memory 62 to realize the preprocessing function of the preprocessing module 16 in which the module board 160 is mounted. The memory 62 is configured to include, for example, a RAM (Random Access Memory), a ROM (Read Only Memory), a flash memory, and a hard disk. The memory 62 stores attribute information to identify the preprocessing function of the preprocessing module 16.

The processor 61 transmits the attribute information read out from the memory 62 to the central control device 80. The central control device 80 receives the attribute information at one of the communication ports 831a to 831k. In this way, the central control device 80 identifies the attribute (preprocessing function) and the mounting area 17 of the preprocessing module 16 mounted on the deck 12. The central control device 80 stores the table data, which includes the attribute and the mounting area 17 of the identified preprocessing module 16, in the memory 82. The central control device 80 performs various types of preprocessing sequentially, based on a predetermined schedule. At this time, the central control device 80 identifies the mounting area 17 on which the preprocessing module 16 capable of performing preprocessing based on the schedule is mounted, based on the table data stored in the memory 82.

### <Processing Procedure of Central Control Device 80>

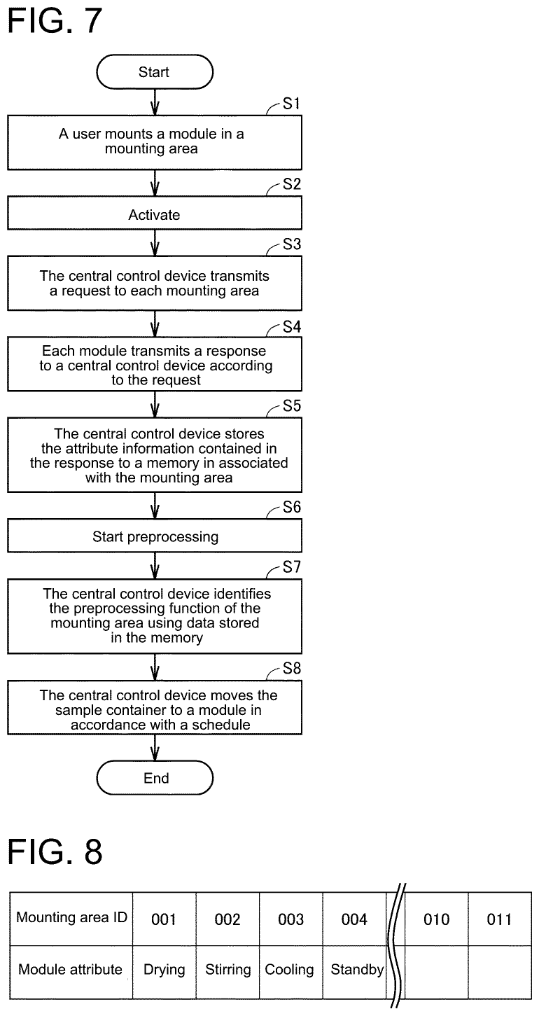

Next, the processing procedures of the central control device 80 and the preprocessing module 16 will be described based on a flowchart. FIG. 7 is a flowchart including the processing performed by the central control device 80 and the preprocessing module 16. FIG. 8 shows the table data stored in the memory 82 of the central control device 80.

Initially, the user mounts a preprocessing module 16 in the mounting area 17 (Step S1). In this embodiment, a plurality of types of preprocessing modules 16 having different preprocessing functions has been prepared in advance. The user selects a necessary preprocessing module 16 according to the application and mounts the selected preprocessing module 16 in one of the mounting areas 17a to 17k. The user may mount the selected preprocessing module 16 in any of the mounting areas 17a to 17k. The number of preprocessing modules 16 required to be mounted in the mounting area differs depending on the application.

The user may set the number of preprocessing modules 16 to 2 (two), 3 (three) or more, depending on the application. The user may mount a plurality of preprocessing modules 16 having the same preprocessing function in any of the mounting areas 17a to 17k. In any case, the central control device 80 automatically determines the location and the attribute (preprocessing function) of the preprocessing module 16 mounted on the deck 12 by the user.

Next, the analysis system 100 is activated in response to the user's operation (Step S2). With this, the preprocessing apparatus 1 included in the analysis system 100 is also activated. Next, the central control device 80 transmits a “request” to each of the mounting areas 17a to 17k using the communication ports 831a to 831k (Step S3). Here, the “request” is a signal that requests the preprocessing module 16 to transmit the attribute information.

The request output from the communication port 831a is directed to the mounting area 17a via the connector 181a and is input to the module board 160 via the connector 171a. Therefore, in the case where the preprocessing module 16 is mounted in the mounting area 17a, the request output from the communication port 831a is received by the preprocessing module 16 mounted in the mounting area 17a.

Next, the preprocessing module 16 mounted in the mounting area 17 transmits a “response” to the central control device 80 in response to the request (Step S4). Here, the “response” is a signal that contains the attribute information on the preprocessing module 16. Among the mounting areas 17a to 17k, the attribute information on the preprocessing modules 16 mounted in the mounting areas 17 is transmitted to the central control device 80 from the mounting areas 17 in which the preprocessing modules 16 are mounted.

Next, the central control device 80 associates the attribute information contained in the response with the position information on the mounting area 17 (any of the mounting areas 17a to 17k) and stores it in the memory 82 (Step S5).

FIG. 8 shows one example of table data stored in the memory 82 in Step S5. The mounting area IDs shown in FIG. 8 are set to correspond to each of the mounting areas 17a to 17k. For example, the “mounting area ID=001” corresponds to the “mounting area 17a” and the “mounting area ID=002” corresponds to the “mounting area 17b.”

In the table data, the attribute information on the preprocessing module 16 is registered by ID (ID=001 to 011) corresponding to each of the mounting areas 17a to 17k. By referring to the table data illustrated in FIG. 8, the central control device 80 identifies that the preprocessing module 16 having a drying function is mounted in the mounting area 17a (ID=0001). Alternatively, by referring to the table data illustrated in FIG. 8, the central control device 80 identifies that the preprocessing module 16 having an agitation function is mounted in the mounting area 17b (ID=0002).

In the table data shown in FIG. 8, no attribute information is registered for each of the mounting areas 17j (ID=0010) and 17k (ID=0011). This means that no preprocessing module 16 is mounted in the mounting areas 17j and 17k. Among the mounting areas 17a to 17k, no response is returned from the mounting area 17 in which the preprocessing module 16 is not mounted. Therefore, the central control device 80 determines that no preprocessing module 16 is mounted in the mounting areas 17 that have not responded to the request.

After completing the processing of Step S5, the central control device 80 starts preprocessing according to a predetermined schedule (Step S6). The central control device 80 that started the preprocessing identifies the preprocessing function of each of the mounting areas 17a to 17k using the table data stored in the memory 82 (Step S7). Next, the central control device 80 moves the sample container 70 to one of the preprocessing modules 16 in the mounting areas 17a to 17k according to the schedule (Step S8). Subsequently, the preprocessing according to the schedule is performed, in the preprocessing module 16.

As described above, according to this embodiment, it is possible to provide the preprocessing apparatus 1 and the analysis system 100, which are capable of handling various types of preprocessing. Here, a conventional preprocessing apparatus, the preprocessing apparatus 1, and the analysis system 100 according to the present embodiment will be compared.

In a conventional preprocessing apparatus, a plurality of hardware components having preprocessing functions was mounted on a deck. However, no conventional preprocessing apparatus was configured so that the hardware components could be exchanged. Therefore, the user could only perform preprocessing within the scope of the preprocessing functions of each of the hardware components mounted on the preprocessing apparatus.

Depending on the purpose of preprocessing, the required preprocessing function for the hardware varies. Further, depending on the purpose of the preprocessing, a plurality of hardware components having the same function may be required. For example, in the preprocessing for a sample A, a plurality of hardware components having a stirring function may be required. In contrast, in the preprocessing for a sample B, no hardware having a stirring function may be required, but hardware having a drying function may be required.

However, there was a problem that a conventional preprocessing apparatus could not adequately address the diversity of the preprocessing purposes. For this reason, the user had to use a plurality of preprocessing apparatuses depending on the preprocessing purpose. Alternatively, the user had to ask a professional contractor to make major modifications that were costly and time-consuming. In this case, the specialized contractor needs to disassemble the hardware of the preprocessing apparatus, install the desired preprocessing mechanism in the preprocessing apparatus, and modify the software of the preprocessing apparatus so that it can recognize the new preprocessing mechanism. As a result, there was a problem that the user's cost burden for modifying the preprocessing apparatus increased.

To solve such a problem, for example, it is conceivable to design the preprocessing apparatus so that the user can freely arrange the hardware components having preprocessing functions along a grid set on the deck.

However, in such a preprocessing apparatus, the user is required to input the information on the position coordinates of the hardware component and the preprocessing function into the preprocessing program each time hardware is added. Similarly, when changing the position of the hardware component, the user is required to input the information on the position coordinates of the hardware component and the preprocessing function into the preprocessing program. This is because inputting the information on the position coordinates and the preprocessing function into the preprocessing program allows the processor of the preprocessing apparatus to identify the position and the function of the hardware placed on the deck.

However, such an input work is not only cumbersome for the user but may also induce errors due to incorrect inputs.

According to this embodiment, the position and the attribute (preprocessing function) of the preprocessing module 16 mounted on the deck 12 by the user are automatically determined by the central control device 80. For this reason, the user is not required to input the location and the attribute of the preprocessing module into the preprocessing program after changing the configuration of the preprocessing module 16 mounted on the deck 12.

Further, according to this embodiment, when the preprocessing module 16 is mounted in the mounting area 17, the preprocessing module 16 is connected to the connector 171 fixed at a predetermined position in the mounting area 17. Therefore, the position of the preprocessing module 16 on the deck 12 is determined by the location of the mounting area 17 where the preprocessing module 16 is mounted. This releases the user from the task of registering the location of the preprocessing module 16 with the central control device 80.

As the coordinate position representing the location of the mounting area 17, the central control device 80 may use the position of the connector 171. The central control device 80 may identify the size of the preprocessing module 16 based on the attribute information on the preprocessing module 16. This also allows the central control device 80 to finely specify the position of the coordinates on the top plate 162.

As described above, according to this embodiment, there is no need to selectively use a plurality of preprocessing apparatuses depending on the purpose of preprocessing. Further, according to this embodiment, there is no need to input information on the mounting area and the preprocessing function of the preprocessing module 16 into the preprocessing program each time hardware related to the preprocessing is added. Therefore, according to this embodiment, it is possible to provide a preprocessing apparatus and an analysis system capable of easily performing an addition or a modification of a preprocessing function.

Note that in this embodiment, from the functional aspect of preprocessing, all types of preprocessing modules 16, including a standby preprocessing module that does not require the module control device 60, are mounted on the module board 160. Even though this is to make the central control device 80 recognize the preprocessing function of the preprocessing module 16, such a configuration could increase the cost of the preprocessing module 16. To keep costs down, for example, it is desirable to standardize the module boards 160 of each of the plurality of types of preprocessing modules 16.

### Modification 1

Next, Modification 1 according to this embodiment will be described. In the embodiment described so far, as many as eleven wires 150 corresponding to the number of mounting areas 17 need to be routed between the deck 12 and the central board 180. Consequently, the design and production of the preprocessing apparatus 1 becomes more complex. Further, it is necessary to provide as many as eleven communication ports corresponding to the number of wiring 150 on the central control device 80. For this reason, the cost required for the central control device 80 will increase.

Therefore, here, the following describes a devise to reduce the number of wiring 150 and the number of communication ports by using a plurality of types of SCI (Serial Communication Interface) signal lines and a plurality of types of Enable signal lines. The central control device 80 transmits a request to the module control device 60 using the SCI signal line. The module control device 60 transmits a response to the central control device 80 using the SCI signal line. The Enable signal lines are used to switch the mounting area in which the SCI signal is enabled among the mounting areas 17a to 17k.

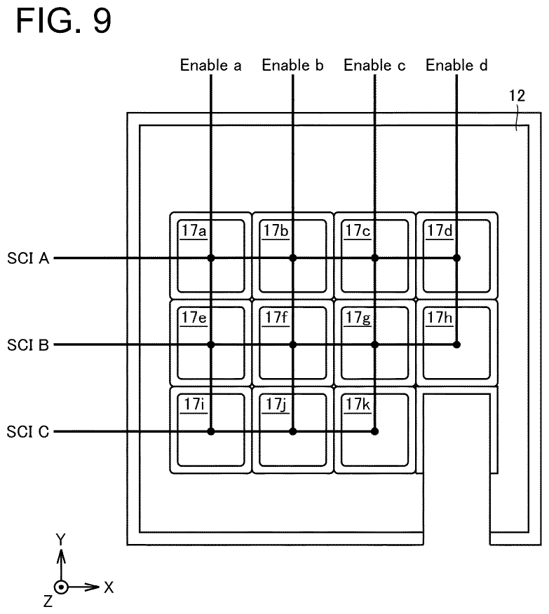

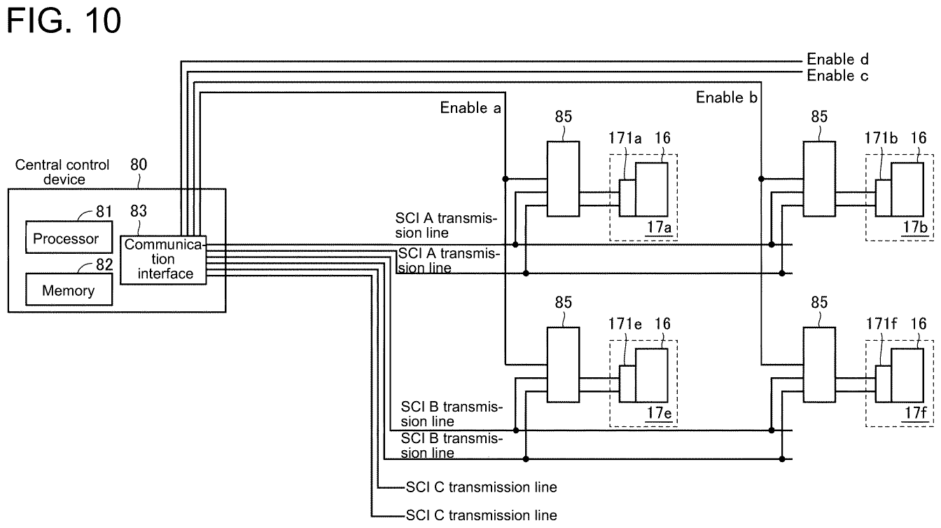

FIG. 9 is a conceptual diagram showing the configuration of Modification 1 regarding the signal lines connecting the mounting areas 17 to the central board 180. FIG. 10 is a block diagram showing the configuration of Modification 1 regarding the signal lines connecting the mounting areas 17 to the central board 180.

FIG. 9 shows an example in which three types of SCI signal lines (SCI A, SCI B, and SCI C) and four Enable signal lines (Enable a, Enable b, Enable c, and Enable d) are wired on the deck 12. As shown in FIG. 9, the mounting areas 17a to 17d are associated with the “SCI A”, the mounting areas 17e to 17h are associated with the “SCI B”, and the mounting areas 17i to 17k are associated with the “SCI C.” Note that in FIG. 9, the illustration of the connector 171 is omitted.

Further, the mounting areas 17a, 17e, and 17i are associated with “Enable a,” the mounting areas 17b, 17f, and 17j are associated with “Enable b,” the mounting areas 17c, 17g, and 17k are associated with “Enable c,” and the mounting areas 17d and 17h are associated with “Enable d.”

In the case where, for example, only “Enable a” is turned on among the four Enable signal lines, “SCI A” forms the communication path between the mounting area 17a and the central board 180, “SCI B” forms the communication path between the mounting area 17e and the central board 180, and “SCI C” forms the communication path between the mounting area 17i and the central board 180.

In the case where, for example, only “Enable b” is turned on among the four Enable signal lines, “SCI A” forms the communication path between the mounting area 17b and the central board 180, “SCI B” forms the communication path between the mounting area 17f and the central board 180, and “SCI C” forms the communication path between the mounting area 17j and the central board 180.

Thus, the communication path is switched by switching the signal line to be turned on among the four types of Enable signal lines. Thus, the central control device 80 can communicate with the number of preprocessing modules 16 corresponding to the product of the number of SCIs and the number of Enable signals.

FIG. 10 shows a block diagram for realizing the configuration shown in FIG. 9. Three types of SCI signal lines and four types of Enable signal lines are connected to the communication interface 83 of the central control device 80. Each of the three types of SCI signal lines is composed of a transmission line and a reception line. In contrast, each of the four types of Enable signal lines is composed of one line. A switching device (communication switching IC) 85 is arranged between the communication interface 83 and each mounting area 17 (17a, 17b . . . ).

Note that the term “transmission line” and “reception line” in FIG. 10 are based on the central control device 80, and the relation between the “transmission” and the “reception” is reversed when based on the preprocessing module 16. This is also true for the term in FIG. 10 and thereafter.

The switching device 85 is mounted on the central board 180. In FIG. 10, the illustration of the central board 180 is omitted. The SCI signal lines (transmission line and reception line) are wired between the central control device 80 and the connectors 171 (171a, 171b . . . ) of the mounting area 17 (17a, 17b . . . ) via the switching device 85. The type of SCI signal line connected to the connector 171 differs depending on the mounting area 17, as shown in FIG. 9.

The Enable signal line is wired between the central control device 80 and the switching device 85. The type of the Enable signal line wired between the central control device 80 and the switching device 85 differs depending on the mounting area 17 corresponding to the switching device 85. This can also be understood by referring to FIG. 9. For example, “Enable a” is wired between the switching device 85 corresponding to the mounting area 17a and the central control device 80. “Enable b” is wired between the switching device 85 corresponding to the mounting area 17b and the central control device 80.

The central control device 80 transmits a request to the SCI signal lines (SCI A, SCI B, and SCI C) while switching the type of Enable signal to be turned on. The switching device 85 opens the communication path configured by the SCI signal line when the Enable signal is turned on and closes the communication path configured by the SCI signal line when the Enable signal is turned off. In other words, the switching device 85 forms a communication path for transmitting attribute information when the Enable signal is turned on.

For example, the preprocessing module 16 mounted in the mounting area 17a receives a request through “SCI A” when “Enable a” is turned on. The preprocessing module 16 mounted in the mounting area 17b receives a request through “SCI A” when “Enable b” is turned on. The preprocessing module 16 mounted in the mounting area 17e receives a request through “SCI B” when “Enable a” is turned on. The preprocessing module 16 mounted in the mounting area 17f receives a request through “SCI B” when “Enable b” is turned on.

The preprocessing module 16 that received a request transmits a response to the central control device 80 using the SCI signal line. Therefore, the central control device 80 can receive a response from the preprocessing module 16 mounted in each mounting area 17. Further, the central control device 80 can identify the mounting location of the preprocessing module 16 that transmitted the response, based on the type of the SCI signal line used to receive the response and the type of the Enable signal that is turned on.

Each of the three types of SCI signal lines is composed of a transmission line and a reception line. In contrast, each of the four types of Enable signal lines is composed of one line. Therefore, as shown in FIG. 10, six SCI signal lines and four Enable signal lines are connected to the communication interface 83 of the central control device 80. In this case, the communication interface 83 requires ten communication ports. In the block diagram shown in FIG. 6, the number of communication ports required for the communication interface 83 is eleven (11). Therefore, according to the configurations shown in FIG. 9 and FIG. 10, the number of communication ports can be reduced as compared with the configuration shown in FIG. 6.

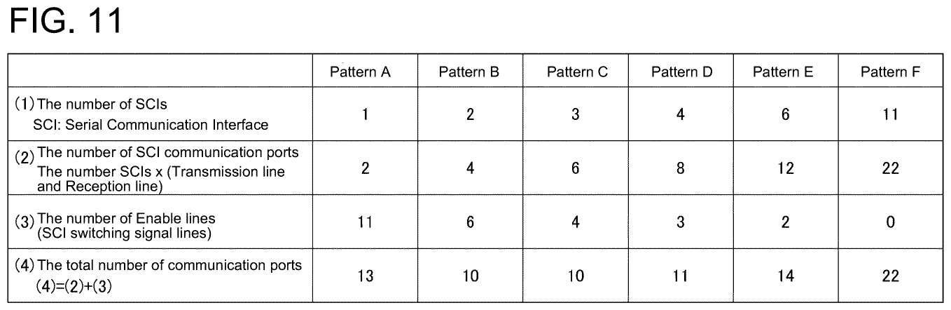

FIG. 11 is a diagram showing the correlation between the number of SCI signal lines and the number of Enable signal lines with respect to Modification 1. In FIG. 9 and FIG. 10, an example is described in which three types of SCI signal lines and four types of Enable signal lines are used. However, each type of the SCI signal line and the Enable signal line is not limited to this. In FIG. 11, an example is shown in which several patterns (Patterns A to F) regarding the combination of SCI signal lines and Enable signal lines that can be employed as Modification 1 are exemplified.

Pattern C corresponds to the configuration shown in FIG. 9 and FIG. 10. In Pattern C, the number of SCIs is 3 (three), so the number of communication ports required for the central control device 80 for SCIs is 6 (six) (3×2). Further, in Pattern C, the number of Enable signal lines is 4 (four). Therefore, the total number of communication ports required for the central control device 80 in Pattern C is 10 (ten).

In Pattern A, the number of SCIs is 1 (one), so the number of communication ports required for the central control device 80 for SCIs is 2 (two). Further, in Pattern A, the number of Enable signal lines is 11 (eleven). Therefore, the total number of communication ports required for the central control device 80 in Pattern A is 13 (thirteen).

In Pattern B, the number of SCIs is 2 (two), so the number of communication ports required for the central control device 80 for SCIs is 4 (four). Further, in Pattern B, the number of Enable signal lines is 6 (six). Therefore, the total number of communication ports required for the central control device 80 in Pattern B is 10 (ten). The total number of communication ports required in Pattern B and the total number of communication ports required in Pattern C are the same. If the designer wishes to reduce the number of SCIs, Pattern B should be employed rather than Pattern C.

In this way, the designer can combine the number of SCI signals and the number of Enable signal lines in various ways. In any case, the central control device 80 can communicate with the number of preprocessing modules 16 corresponding to the product of the number of SCIs and the number of Enable signals. In particular, if the designer wishes to reduce the number of wiring 150 and the number of communication ports compared with the configuration shown in FIG. 9 and FIG. 10, Pattern B or Pattern C should be adopted.

In Modification 1, the SCI signal line is one example of a communication line, and the Enable signal line is one example of a control line.

### Modification 2

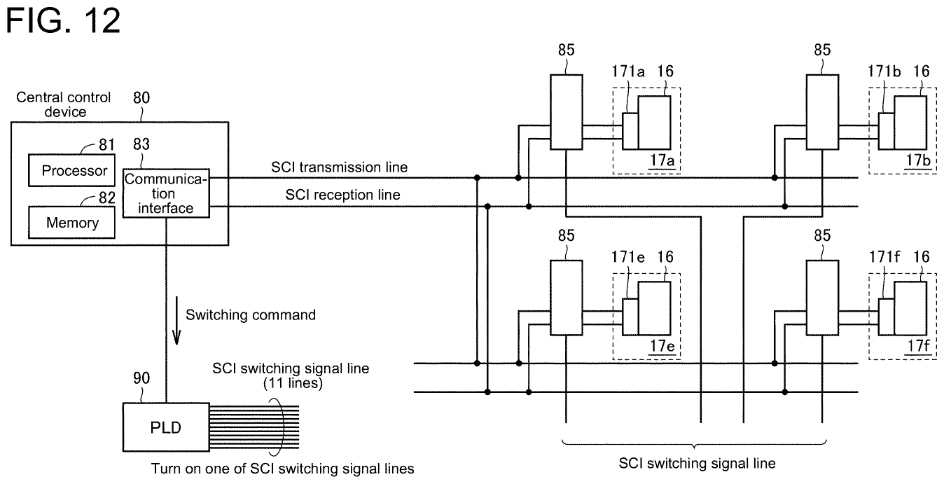

Next, Modification 2 according to this embodiment will be described. FIG. 12 is a block diagram showing the configuration according to Modification 2. In Modification 2, a PLD (programmable logic device) 90 is adopted to reduce the number of communication ports required for the central control device 80, compared with the configuration of Modification 1. The PLD 90 is provided, for example, on the central board 180.

As shown in FIG. 12, the communication interface 83 of the central control device 80 is connected to the PLD 90 by a single wire. The PLD 90 is one example of a device that expands the number of communication ports of the central control device 80. The PLD 90 and the switching devices 85 corresponding to the respective mounting areas 17 (17a, 17b . . . ) are connected by the SCI switching signal lines.

A pair of SCI signal lines (SCI transmission line and SCI reception line), which constitute the SCI, are connected to the communication interface 83. The pair of signal lines constituting the SCI are connected to the switching device 85 corresponding to the respective mounting areas 17.

The central control device 80 transmits a switching command to the PLD 90 via the communication interface 83. The switching command is information to notify which of the communication paths toward each of the mounting areas 17 is to be enabled. The PLD 90 turns on the SCI switching signal line corresponding to the switching command. This allows the central control device 80 to communicate with the preprocessing module 16 mounted in each of the mounting areas 17 using a pair of SCI signal lines. According to the configuration of Modification 2, the number of communication ports required for the communication interface 83 can be reduced to 3 (three). Further, according to Modification 2, it is possible to reduce the burden on the central control device 80 in controlling the signal state of the SCI signal switching signal lines.

In Modification 2, the SCI signal lines are one example of the communication lines, the SCI switching signal lines are one example of the control lines, and the PLD 90 is one example of a third switching device.

In Modification 1 and Modification 2, the switching device 85 connected to the connector 171a is one example of a first switching device, and the switching device 85 connected to the connector 171b is one example of a second switching device. In Modification 1 and Modification 2, the SCI signal lines are one example of communication lines. In Modification 1, the Enable signal line (Enable a) is one example of a first control line, and the Enable signal line (Enable b) is one example of a second control line. In Modification 2, the SCI switching signal line corresponding to the “connector 171a” is one example of a first control line, and the SCI switching signal line corresponding to the “connector 171b” is one example of a second control line. In Modification 2, the PLD 90 is one example of a signal switching device.

### Modification 3

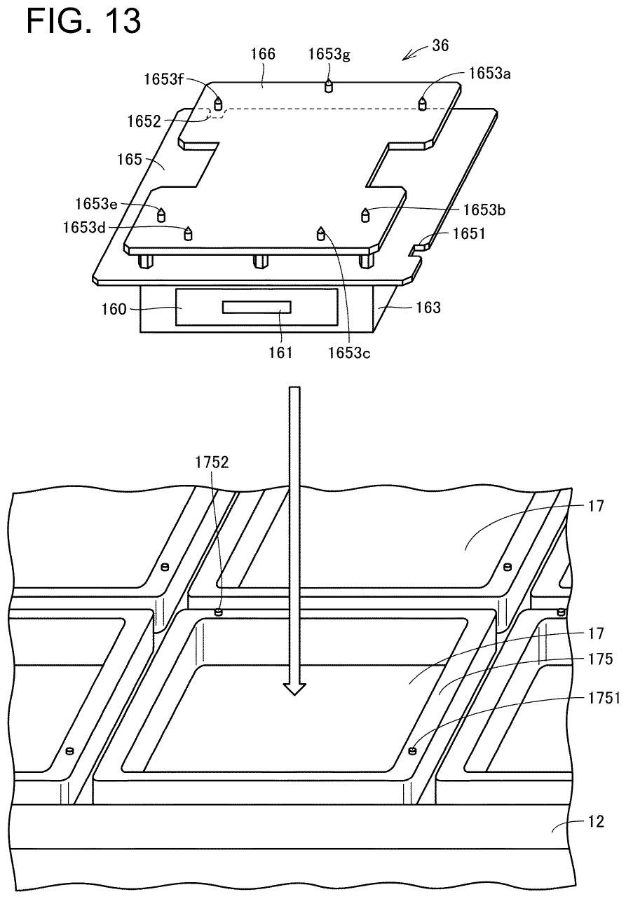

Next, referring to FIG. 13 to FIG. 16, Modification 3 of this embodiment will be described. In Modification 3, a method to eliminate the need for teaching when preprocessing modules are mounted in the mounting area 17 will be described.

Teaching is the task of informing the central control device 80 of the mounting position of the preprocessing module. According to this embodiment, the central control device 80 can automatically identify the mounting area 17 of the preprocessing module 16 by communicating with the module board 160. Therefore, in this embodiment, no teaching is required to inform the central control device 80 of the mounting area 17 of the preprocessing module 16.

However, the central control device 80 cannot finely control the position information of the gripper arm 14 with respect to the top plate 162 of the preprocessing module 16 using only the information on the mounting area of the preprocessing module 16. For this reason, a reference position for the gripper arm 14 is set on the top plate 162. However, when assembling the preprocessing module 16, the reference position may be misaligned due to individual differences in the components. Considering the occurrence of such misalignment, teaching is required to compensate for the misalignment when the preprocessing module 16 is mounted in the mounting area 17.

Teaching is a time-consuming task, although it is usually performed by an experienced service person. In cases where such teaching is required for each of the preprocessing modules 16, the working time will become even longer. If a teaching error occurs due to human error by a service person, parts may contact each other in the preprocessing apparatus, or other problems may occur. Therefore, in Modification 3, a method to eliminate the need for such teaching is proposed.

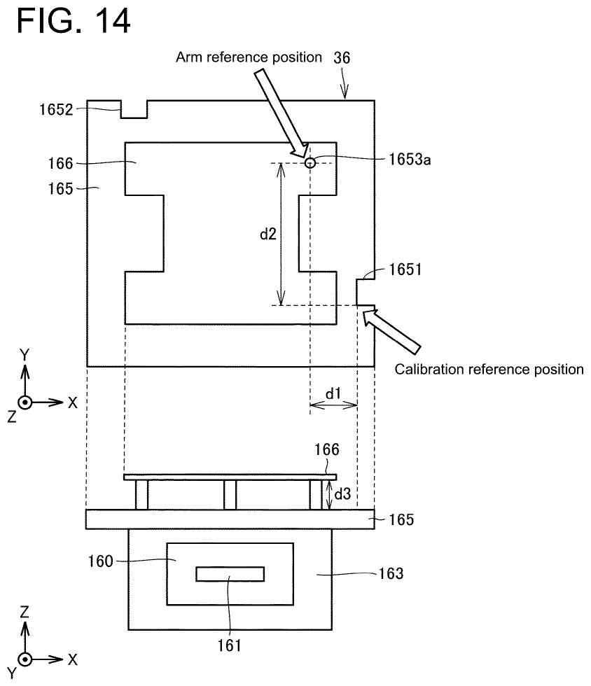

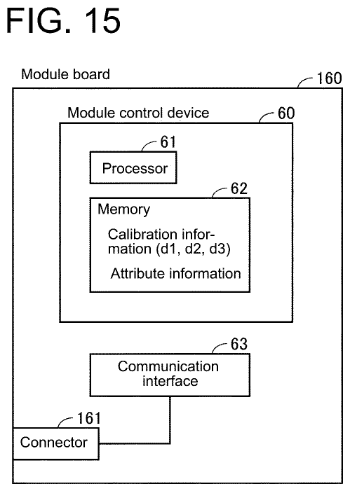

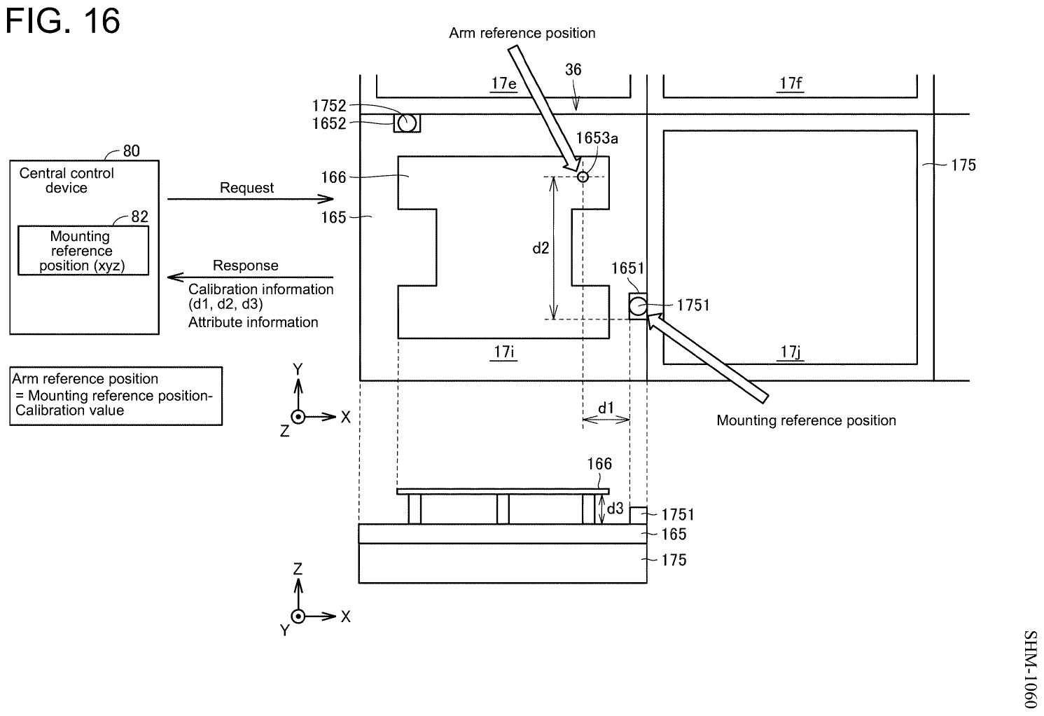

FIG. 13 is a diagram showing the relation between the preprocessing module 36 according to Modification 3 and the mounting area 17. FIG. 14 is a diagram showing the mounting reference position and the arm reference position set in the preprocessing module. FIG. 15 is a block diagram showing the configuration of the module board 160 of Modification 3. FIG. 16 is a conceptual diagram for explaining the information to be transmitted from the preprocessing module 36 to the central control device 80.

As shown in FIG. 13, the preprocessing module 36 of Modification 3 is provided with a support base 163, a module board 160, a first member 165, and a second member 166. The first member 165 and the second member 166 are plate-shaped members. A module board 160 having a connector 161 is attached to the side of the support base 163, and the first member 165 is attached to the top of the support base 163. The second member 166 is attached to the top of the first member 165.

The preprocessing module 36 is mounted in the mounting area 17, similar to the preprocessing module 16 shown in FIG. 4. Reference projections 1751 and 1752 are provided on the mounting frame 175 surrounding the mounting area 17. The first member 165 is provided with notches 1651 and 1652. The user adjusts the position of the preprocessing module 36 so that the reference projections 1751 and 1752 fit into the notches 1651 and 1652, respectively, when mounting the preprocessing module 36 in the mounting area 17. With this, the preprocessing module 36 is placed in a predetermined position in the mounting area 17. As described above, in Modification 3, the mounting reference position of the preprocessing module 36 is determined by the reference projections 1751 and 1752.

The second member 166 of the preprocessing module 36 corresponds to the top plate 162 of the preprocessing module 16 shown in FIG. 3. Therefore, the sample container 70 is placed on the second member 166. The second member 166 has positioning projections 1653a to 1653g for determining the position of the sample container 70.

Further, the second member 166 has a pre-set arm reference position. The central board 180 controls the position of the gripper arm 14 relative to the preprocessing module 36, based on the arm reference position. The arm reference position can be set in various positions, but here, for example, it is assumed that the position of the positioning projection 1653a is set as the arm reference position.

In FIG. 14, the calibration reference position is shown in which the notch 1651 is used as a representative example of the notches 1651 and 1652. As shown in FIG. 14, here it is defined that the distance from the calibration reference position to the arm reference position in the X-axis direction is d1, the distance from the calibration reference position to the arm reference position in the Y-axis direction is d2, and the distance from the calibration reference position to the arm reference position in the Z-axis direction is d3.

The central board 180 stores the coordinates (x, y, z) of the reference projection 1751 of each mounting area 17 on the deck 12. If the distances d1 to d3 shown in FIG. 14 and the coordinates of the reference projections 1751 are known, the central board 180 can calculate the arm reference position of the preprocessing module 36 mounted in the mounting area 17. However, in the process of assembling the second member 166 to the first member 165, variations from the designed values may occur in each of the distances d1 to d3.

In this case, an error occurs between the arm reference position of the preprocessing module 36 calculated based on the design values d1 to d3 and the arm reference position of the preprocessing module 36 actually mounted in the mounting area 17. If such an error occurs, the central board 180 cannot accurately control the gripper arm 14.

Therefore, in the process of assembling the second member 166 to the first member 165, the worker measures the distances d1 to d3 and registers the measured values as calibration information on the module board 160. The worker is, for example, a factory worker who assembles the preprocessing module 36. In Modification 3, the central board 180 calculates the arm reference position using the coordinates of the reference projection 1751 and the calibration information (d1, d2, d3) registered on the module board 160.

As shown in FIG. 15, calibration information (d1, d2, d3) is stored in the memory 62 of the module board 160, in addition to attribute information.

In FIG. 16, an example is shown in which a preprocessing module 36 is mounted in the mounting area 17i. The central control device 80 transmits a request to the preprocessing module 36 according to a procedure similar to that of the flowchart shown in FIG. 7. The module control device 60 mounted on the preprocessing module 36 reads out the calibration and attribute information from the memory 62 and transmits the read calibration information and attribute information to the central control device 80.

In the memory 82 of the central control device 80, the coordinates of the mounting reference position are stored for each mounting area 17. The central control device 80 calculates the arm reference position of the preprocessing module 36 mounted in the mounting area 17i, based on the mounting reference position of the mounting area 17i and the calibration value based on the calibration information. The central control device 80 controls the gripper arm 14 with high accuracy, based on the calculated arm reference position.

According to Modification 3, the man-hours required for assembling the preprocessing module 36 will increase, but the man-hours required for the service personnel working under the user will be greatly reduced. As a result, the number of man-hours involved from the user's perspective can be greatly reduced. Furthermore, according to Modification 3, human errors caused by teaching are eliminated, thus preventing problems such as parts colliding with each other in the preprocessing apparatus 1 due to human error caused by teaching.

Note that here an example is shown in which the distances d1 to d3 are measured after the second member 166 is assembled to the first member 165. However, the second member 166 may be assembled to the first member 165 using a jig so that the distances d1 to d3 match the predetermined calibration values. Further, in Modification 3, the preprocessing module 36 may transmit the position information (coordinates) of the preprocessing module 36 mounted on the deck 12 to the central control device 80, along with the calibration information and the attribute information. The preprocessing module 36 may, for example, transmit the mounting reference position of the mounting area 17 where the preprocessing module 36 is placed to the central control device 80 as position information.

### Other Modifications

In the above-described embodiments, an example is shown in which the connector 171 mounted in the mounting area 17 and the central control device 80 are connected by wiring. In this case, the central control device 80 and the preprocessing module 16 mounted in the mounting area 17 perform wired communication. However, it may be configured such that the central control device 80 and the preprocessing module 16 mounted in the mounting area 17 may communicate wirelessly. In this case, the wiring 150 shown in FIG. 5 can be eliminated.

For example, a first wireless communication device having a connector 171 may be placed in each mounting area 17. In this case, the first wireless communication device placed in the mounting area 17a is one example of the first communication unit placed in the first mounting area. Further, the first wireless communication device placed in the mounting area 17b is one example of the second communication unit placed in the second mounting area. In this case, a second wireless communication device that communicates with the first wireless communication device is placed on the central board 180 instead of the connector 181.

### Aspects

It should be understood by those skilled in the art that the above-described embodiments and modifications thereof are specific examples of the following aspects.

### (Item 1)

A preprocessing apparatus according to one aspect of the present disclosure is a preprocessing apparatus (1) for preprocessing a sample contained in a sample container (70).

The preprocessing apparatus includes:
a first preprocessing module (16a) having a first function related to the preprocessing, the first preprocessing module being configured to output first attribute information for identifying the first function;
an arm (141) configured to transfer the sample container to the first processing module;
a mounting unit (12) having a first mounting area (17a) and a second mounting area (17b), each of the first mounting area (17a) and the second mounting area (17b) being configured to mount the first preprocessing module therein;
a central control device (80);
a first communication unit (171a) arranged in the first mounting area; and
a second communication unit (171b) arranged in the second mounting area,
wherein the first preprocessing module transmits the first attribute information to the central control device via the first communication unit when the first preprocessing module is mounted in the first mounting area (Step S4),
wherein the first preprocessing module transmits the first attribute information to the central control device via the second communication unit when the first preprocessing module is mounted in the second mounting area (Step S4), and
wherein the central control device is configured to
identify that the first preprocessing module is mounted in the first mounting area when the first attribute information is received from the first communication unit (Step S7), and
identify that the first preprocessing module is mounted in the second mounting area when the first attribute information is received from the second communication unit (Step S7).

According to the preprocessing apparatus as recited in the above-described Item 1, it is possible to provide a preprocessing apparatus that can easily implement addition or modification of a preprocessing function.

### (Item 2)

In the preprocessing apparatus as recited in the above-described Item 1, it may be configured such that the preprocessing apparatus as recited in the above-described Item 2 further includes:
a second preprocessing module (16b) having a second function related to the preprocessing, the second preprocessing module being configured to output second attribute information for identifying the second function,
wherein the second preprocessing module transmits the second attribute information to the central control device via the first communication unit when the second preprocessing module is mounted in the first mounting area (Step S4),
wherein the second preprocessing module transmits the second attribute information to the central control device via the second communication unit when the second preprocessing module is mounted in the second mounting area (Step S4), and
wherein the central control device is configured to
identify that the second preprocessing module is mounted in the first mounting area when the second attribute information is received from the first communication unit (Step S7), and
identify that the second preprocessing module is mounted in the second mounting area when the second attribute information is received from the second communication unit (Step S7).

According to the preprocessing apparatus as recited in the above-described Item 2, it is possible to provide a preprocessing apparatus that can easily implement the addition or modification of a preprocessing function.

### (Item 3)

In the preprocessing apparatus as recited in the above-described Item 1 or 2, it may be configured such that in the preprocessing apparatus as recited in the above-described Item 3,
the central control device includes a communication interface (83) provided with a first port (831a) and a second port (831b), and
the central control device communicates with the first communication unit via the first port and communicates with the second communication unit via the second port.

According to the preprocessing apparatus as recited in the above-described Item 3, the central control device can identify the mounting area where the first preprocessing module is mounted by identifying the port where attribute information is input.

### (Item 4)

In the preprocessing apparatus as recited in any one of the above-described Items 1 to 3, it may be configured such that in the preprocessing apparatus as recited in the above-described Item 4,
each of the first communication unit and the second communication unit is a connector,
the first preprocessing module includes:
a module control device (60); and
a module connector (161) connected to the module control device,
each of the first communication unit and the second communication unit is connected to the central control device by wiring (150),
when the first preprocessing module is mounted in the first mounting area, the module connector is connected to the first communication unit, and
when the first preprocessing module is mounted in the second mounting area, the module connector is connected to the second communication unit.

According to the preprocessing apparatus as recited in the above-described Item 4, the central control device and the module control device are connected by a wired communication path, which allows more reliable communication between the central control device and the module control device.

### (Item 5)

In the preprocessing apparatus as recited in the above-described Item 5, it may be configured such that the preprocessing apparatus as recited in the above-described Item 4 further includes:
a first switching device (85) connected to the first communication unit; and
a second switching device (85) connected to the second communication unit,
wherein the central control device is connected to the first communication unit via the first switching device (see FIG. 10 and FIG. 12),
wherein the central control device is connected to the second communication unit via the second switching device (FIG. 10 and FIG. 12),
wherein the wiring includes:
a communication line (SCI signal line) forming a communication path for transmitting the first attribute information;
a first control line (Enable signal line (Enable a) SCI switching signal line) connected to the first switching device; and
a second control line (Enable signal line (Enable b), SCI signal switching line)) connected to the second switching device,
wherein the first communication unit and the second communication unit are connected in parallel to the communication line (FIG. 10 and FIG. 12),
wherein the central control device is configured to switch a signal state of each of the first control line and the second control line such that when the signal state of the first control line is in a first state, the signal state of the second control line is in a second state, and when the signal state of the first control line is in the second state, the signal state of the second control line is in the first state (FIG. 10 and FIG. 12),
wherein the first switching device forms the communication path between the first communication unit and the central control device when the signal state of the first control line is in the first state (FIG. 10 and FIG. 12), and
wherein the second switching device forms a communication path between the second communication unit and the central control device when the signal state of the second control line is in the first state (FIG. 10 and FIG. 12).

According to the preprocessing apparatus as recited in the above-described Item 5, it is possible to share the same communication path for communicating the first attribute information to the first communication unit and the communication path for communicating the first attribute information to the second communication unit.

### (Item 6)

In the preprocessing apparatus as recited in the above-described Item 5, it may be configured such that the preprocessing apparatus as recited in the above-described Item 6 further includes:
a signal switching device (90),
wherein the first control line is wired between the signal switching device and the first switching device (FIG. 12),
wherein the second control line is wired between the signal switching device and the second switching device (FIG. 12),
wherein the central control device transmits a command for switching the signal state of each of the first control line and the second control line to the signal switching device (FIG. 12), and
wherein the signal switching device switches the signal state of each of the first control line and the second control line based on the command (FIG. 12).

According to the preprocessing apparatus as recited in the above-described Item 6, it is possible to reduce the burden on the central control device to control the respective signal states of the first control line and the second control line by means of the signal switching device.

### (Item 7)

In the preprocessing apparatus as recited in any one the above-described Items 1 to 6, it may be configured such that in the preprocessing apparatus as recited in the above-described Item 7,
the first preprocessing module includes:
a first member (165) to be mounted at a reference position in the first mounting area (1751);
a second member (166) having a placement surface for the sample container, and
a memory (62) storing calibration information for calibrating a positional relation between the first member and the second member,
wherein when the first preprocessing module is mounted in the first mounting area, the first preprocessing module transmits the calibration information to the central control device via the first communication unit (FIG. 16), and
wherein the central control device calibrates a reference position of the arm defined on the mounting surface based on the calibration information (FIG. 16).

According to the preprocessing apparatus as recited in the above-described Item 7, it is possible to eliminate the need for the teaching work related to the arm reference position when the first preprocessing module is mounted on the preprocessing apparatus.

### (Item 8)

An analysis system as recited in the above-described Item 8 includes the preprocessing apparatus as recited in any one of the above-described Items 1 to 7.

According to the analysis system as recited in the above-described Item 8, it is possible to provide an analysis system capable of easily implementing additions or modifications to a preprocessing function.

Although some embodiments of the present invention have been described, the embodiments disclosed here should be considered in all respects illustrative and not restrictive. It should be noted that the scope of the invention is indicated by claims and is intended to include all modifications within the meaning and scope of the claims and equivalents.

1. A preprocessing apparatus for performing preprocessing of a sample contained in a sample container, comprising:
a first preprocessing module having a first function related to the preprocessing, the first preprocessing module being configured to output first attribute information for identifying the first function;
an arm configured to transfer the sample container to the first preprocessing module;
a mounting unit having a first mounting area and a second mounting area, each of the first mounting area and the second mounting area being configured to mount the first preprocessing module therein;
a central control device;
a first communication unit arranged in the first mounting area; and
a second communication unit arranged in the second mounting area,
wherein the first preprocessing module transmits the first attribute information to the central control device via the first communication unit when the first preprocessing module is mounted in the first mounting area,
wherein the first preprocessing module transmits the first attribute information to the central control device via the second communication unit when the first preprocessing module is mounted in the second mounting area, and
wherein the central control device is configured to
identify that the first preprocessing module is mounted in the first mounting area when the first attribute information is received from the first communication unit, and
identify that the first preprocessing module is mounted in the second mounting area when the first attribute information is received from the second communication unit.

2. The preprocessing apparatus as recited in claim 1, further comprising:
a second preprocessing module having a second function related to the preprocessing, the second preprocessing module being configured to output second attribute information for identifying the second function,
wherein the second preprocessing module transmits the second attribute information to the central control device via the first communication unit when the second preprocessing module is mounted in the first mounting area,
wherein the second preprocessing module transmits the second attribute information to the central control device via the second communication unit when the second preprocessing module is mounted in the second mounting area, and
wherein the central control device is configured to
identify that the second preprocessing module is mounted in the first mounting area when the second attribute information is received from the first communication unit, and
identify that the second preprocessing module is mounted in the second mounting area when the second attribute information is received from the second communication unit.

3. The preprocessing apparatus as recited in claim 1,
wherein the central control device includes a communication interface provided with a first port and a second port, and
wherein the central control device communicates with the first communication unit via the first port and communicates with the second communication unit via the second port.

4. The preprocessing apparatus as recited in claim 1,
wherein each of the first communication unit and the second communication unit is a connector,
wherein the first preprocessing module includes:
a module control device; and
a module connector connected to the module control device,
wherein each of the first communication unit and the second communication unit is connected to the central control device by wiring,
wherein when the first preprocessing module is mounted in the first mounting area, the module connector and the first communication unit are connected to each other, and
wherein when the first preprocessing module is mounted in the second mounting area, the module connector and the second communication unit are connected to each other.

5. The preprocessing apparatus as recited in claim 4, further comprising:
a first switching device connected to the first communication unit; and
a second switching device connected to the second communication unit,
wherein the central control device is connected to the first communication unit via the first switching device,
wherein the central control device is connected to the second communication unit via the second switching device,
wherein the wiring includes:
a communication line forming a communication path for transmitting the first attribute information;
a first control line connected to the first switching device; and
a second control line connected to the second switching device,
wherein the first communication unit and the second communication unit are connected in parallel to the communication line,
wherein the central control device is configured to switch a signal state of each of the first control line and the second control line such that when the signal state of the first control line is in a first state, the signal state of the second control line is in a second state, and when the signal state of the first control line is in the second state, the signal state of the second control line is in the first state,
wherein the first switching device forms the communication path between the first communication unit and the central control device when the signal state of the first control line is in the first state, and
wherein the second switching device forms the communication path between the second communication unit and the central control device when the signal state of the second control line is in the first state.

6. The preprocessing apparatus as recited in claim 5, further comprising:
a signal switching device,
wherein the first control line is wired between the signal switching device and the first switching device,
wherein the second control line is wired between the signal switching device and the second switching device,
wherein the central control device transmits a command for switching the signal state of each of the first control line and the second control line to the signal switching device, and
wherein the signal switching device switches the signal state of each of the first control line and the second control line based on the command.

7. The preprocessing apparatus as recited in claim 1,
wherein the first preprocessing module includes:
a first member to be mounted at a reference position in the first mounting area,
a second member having a placement surface for the sample container, and
a memory storing calibration information for calibrating a positional relation between the first member and the second member,
wherein when the first preprocessing module is mounted in the first mounting area, the first preprocessing module transmits the calibration information to the central control device via the first communication unit, and
wherein the central control device calibrates a reference position of the arm defined on the mounting surface based on the calibration information.

8. An analysis system comprising the preprocessing apparatus as recited in claim 1.
